(19) United States
(12) Patent Application Publication
(10) Pub. No.: US 2019/0117467 A1
(43) Pub. Date: Apr. 25, 2019

(54) METHOD AND DEVICE FOR PROMOTING FUNCTIONAL RESTORATION OF A USER BODY PART

(71) Applicant: Yvonne Darlene Cagle, Cambell, CA (US)

(72) Inventor: Yvonne Darlene Cagle, Cambell, CA (US)

(21) Appl. No.: 16/168,557

(22) Filed: Oct. 23, 2018

Related U.S. Application Data
(60) Provisional application No. 62/576,285, filed on Oct. 24, 2017.

(51) Int. Cl. A61F 13/10; A61F 13/08

(52) U.S. Cl. CPC A61F 13/10; A61B 2017/00862; A61F 13/08

(57) ABSTRACT

A method and device for promoting functional restoration of a user body part, having a material capable of generating a distributed pressure, within a pressure range, to the user body part in a concentric design to relieve pain, amplify strength and accelerate functional restoration, in the field of therapy, apparatus for physical training and medicine.

### STATEMENT OF RELATED APPLICATIONS

This application claims the benefit of and priority on U.S. Provisional Patent Application No. 62/576,285 having a filing date of 24 Oct. 2017.

### BACKGROUND OF THE INVENTION

### Technical Field

The present invention refers to a method and a device for promoting functional restoration of a user body part, based on generating a distributed pressure to the user body part, within a predefined pressure range, to the user body part in a concentric design to relieve pain, amplify strength and accelerate functional restoration. The present invention is situated in the field of therapy, apparatus for physical training and medicine.

### Prior Art

The body's natural ability to heal itself can be improved in a way to reduce the time needed to heal the injured, inflamed sprained tissue, strained and sprained tissue.

The state of the art requires devices and methods for an efficient acceleration on the healing process of injured limbs.

As can be inferred from literature, there are no documents suggesting or anticipating the teachings of the present invention, so that the solution proposed here has novelty and inventive step outside the state of the art.

### BRIEF SUMMARY OF THE INVENTION

The present invention provides a method and device for promoting functional restoration of a user body part, based on generating a distributed pressure to the user body part, within a pressure range, to relieve pain, amplify strength and accelerate functional restoration. Further, the method can reduce the symptoms of myofascial plane imbalances or deconditioning.

In a first aspect, the present invention provides a method for promoting functional restoration of a user body part comprising generating a distributed pressure by means of a device to the user body part, wherein said distributed pressure is provided by at least a force applied by the device.

In a second aspect, the present invention provides a device for promoting functional restoration of a user body part, wherein the device comprises a material comprising an intrinsic property for generation of a distributed pressure to the user body part, wherein said distributed pressure is provided by at least a force applied by the material in at least one direction.

These and other aspects of the invention will be immediately appreciated by the well versed in the art, and for companies with interests in the product segment and will be described in sufficient detail to be reproduced in the following description.

### BRIEF DESCRIPTION OF THE DRAWINGS

This invention presents the following drawings in order to define an exemplary embodiment of the proposed system and method however, without limiting the scope of protection.

FIG. 1 shows an embodiment of the electronic sensor and circuit connected to the surface of a material of the device.

FIG. 2 shows a flowchart of an embodiment of an electronic sensor connected to an electronic circuit.

FIG. 3 shows an embodiment of the electronic circuit.

### DETAILED DESCRIPTION OF PREFERRED EMBODIMENTS

In a first aspect, the present invention provides a method for promoting functional restoration of a user body part comprising generating a distributed pressure by means of a device to the user body part, wherein said distributed pressure is provided by at least a force applied by the device.

The step of generating a distributed pressure is performed by a device adapted to generate a force and hence a distributed pressure throughout its area. Thus, the step of generating a distributed pressure comprises applying a force in at least one extremity or end of said device, in order to reach a predefined pressure range, and attaching said device submitted to the force in the user body part. In this sense, the device is able to receive a force and to transfer partially or totally said force to the user body part.

In an embodiment, the device used to generate a distributed pressure comprises an elastic property in order to allow storing energy through strain, e.g., stretching and compressing. In an embodiment, said device comprises an elastic material in at least one portion of the device. In an embodiment, the device comprises an elastic material forming the whole area of the device.

For the present invention, the term “elastic material” comprises any material capable of storing the energy used to contract, stretch, or distort said material and resume to a non-stressed configuration. In this sense, the elastic material has been developed to be attached and to apply a pressure in the user body part.

The step of generating a distributed pressure further comprises applying a force in at least one extremity of said device, in which the force is applied in order to reach a predefined pressure range. In an embodiment, the force is applied in order to reach a predefined pressure range and to stretch the device, in such a way that the stretched device is attached to the user body part. In an embodiment, the device is stretched since it comprises a resilience property, and therefore it tends to return to a non-stressed configuration, wherein the non-stressed configuration is a natural static state. In an embodiment, the device comprises a material being an elastic material with a stiffness coefficient, where the device when stretched performs a distributed pressure directed towards a neutral axis. In this sense, the distributed pressure is partially or totally transferred to the user body part.

In another embodiment, the step of generating a distributed pressure further comprises applying a force on the device to attach said device in the user body part. In one embodiment, the step of generating a distributed pressure further comprises a reaction of chemical reagents in order to generate said distributed pressure, within the predefined pressure range, after the attaching of the device in the user body part. For example, the device is comprised by a material that is able to be stretched and retracted by chemical reactions. Furthermore, in another embodiment, the device comprises a material provided with a coefficient of thermal expansion, where it is stretched by thermal expansion and retracted by reverse effect.

In one embodiment, the step of generating a distributed pressure further comprises electrical transduction in the device in order to generate said distributed pressure, within the predefined pressure range, after the attaching of the device in the user body part, wherein the electrical transduction converts electrical signals into the distributed pressure. In a further embodiment, the device comprises microelectromechanical systems (MEMS), such as a telescopic mechanism in a plan sheet geometry, where it comprises low thickness and can be attached in the user body part. Therefore, when an electric current is applied the MEMS are able to be retracted or stretched.

In an embodiment, the predefined pressure comprises a pressure range of 1 mmHg to 150 mmHg. In another embodiment, the predefined pressure comprises a pressure range of 3 mmHg to 130 mmHg. Therefore, the compression force applied by the device is titrated to comfort the user and improves blood circulation.

In an embodiment, the applying of the force goes on until it reaches a predefined pressure. After the device is attached to the user body part, the device transfers the predefined pressure, totally or partially, to the user body part, in such a way to distribute the pressure in the user body part over the area where the device is attached.

In an embodiment, the applying of the force comprises the stretching of the device in at least one extremity of the device in order to generate a predefined pressure, and then attached to the user body part. The generated pressure on the user body part is directed towards the neutral axis of the device. Therefore, in an embodiment, the extremities of the device tend to concentrate the strain of the user body part.

In one embodiment, the step of generating a distributed pressure by means of a device to the user body part generates a measured pressure, which is identified by a component for indication of pressure. In one embodiment, the applying a force in at least one extremity of said device generates a measured pressure, which is identified by a component for indication of pressure. In another embodiment, the reaction of chemical reagents generates a measured pressure, which is identified by a component for indication of pressure. In another embodiment, the electrical transduction in the device generates a measured pressure, which is identified by a component for indication of pressure.

In a further embodiment, the component for indication of pressure is a visual indicator. The visual indicator is positioned in the device and indicates different pressures according to the force applied in at least one extremity of the device by changing its format along the pressure applied in the device. In an embodiment, the visual indicator is positioned in the device and indicates different pressures according to the reaction of chemical reagents. In an embodiment, the visual indicator is positioned in the device and indicates different pressures according to the electrical transduction in the device.

In an example, the visual indicator is any kind of drawings positioned in one surface of the device, and said drawing varies according to the pressure changing.

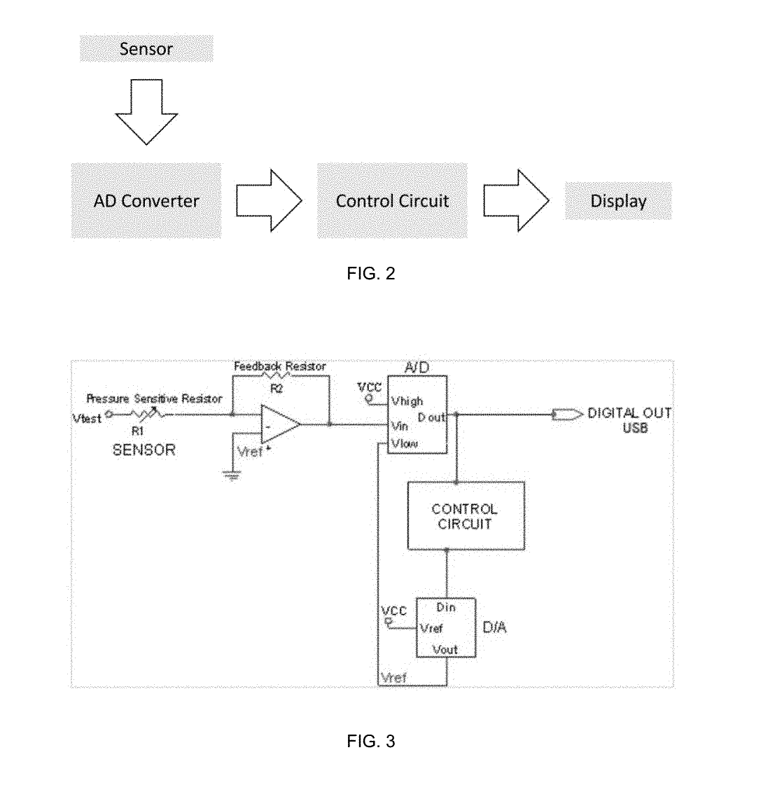

In another embodiment, the component for indication of the measured pressure is an electronic circuit 3, wherein the electronic circuit 3 is connected to an electronic sensor 2 arranged to detect the applied force in at least one extremity of the device, the electronic circuit 3 process the detected force and displays the pressure to be applied by the device, as it can be seen on the flowchart of FIG. 2. In an embodiment, the electronic sensor 2 detects the generated pressure of the reaction of chemical reagents. In an embodiment, the electronic sensor 2 detects the generated pressure of the electrical transduction.

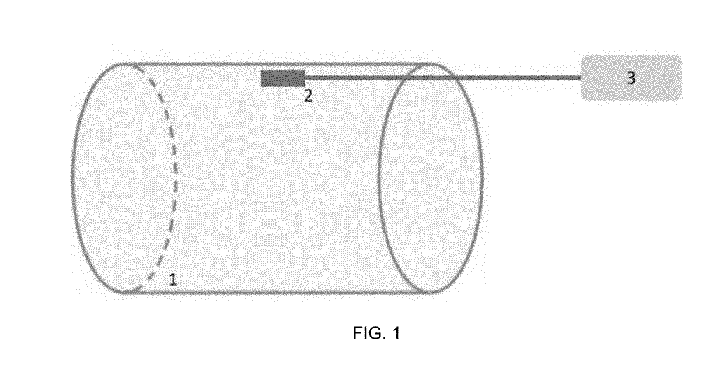

In an example, the electronic sensor 2 is positioned on the outer surface of the device, in such a way to detect the strain in which the device is submitted, as shown by FIG. 1.

To further improve the functional restoration, the device is attached to the user body part in a concentric design. In another embodiment, a plurality of devices is harmonically arranged by concentric nulling of loads on the user body part.

In an embodiment, the method as provided in the present invention is applied in upper and lower extremities of human body, in order to promote the acceleration and amplification of strength and functional restoration resulting from myofascial plane imbalances or deconditioning. In another embodiment, the user body part is a limb of an animal, for example, a horse, a dog, a cat, etc.

In a second aspect, the present invention provides a device for promoting functional restoration of a user body part, wherein the device comprises a material comprising an intrinsic property for generation of a distributed pressure to the user body part, wherein said distributed pressure is provided by at least a force applied by the material in at least one direction.

In an embodiment, the device comprises a structure comprising at least one of the following, but not limited to: knitted material; crocheted material; quilted material; cross cross-stitched sleeve glove or sock; flexible weave; leg-warmer; support hosiery; compressible garment; stretchable material; corset; cotton material; polyester material; nylon material; neoprene material; comprehensible plastic; saran material; and shearable washable textile.

In an embodiment, the device comprises a configuration of property comprising at least one of the following, but not limited to: wearable, stretchable; water-resistant; breathable; soft; portable; and moldable.

In an embodiment, the intrinsic property comprises a compression force related to an applied force in at least one extremity of the material, wherein the compression force generates a distributed pressure within a pressure range. In another embodiment, the intrinsic property comprises a compression force related to a reaction of chemical reagents in the material, wherein the compression force generates a distributed pressure within a pressure range. In another embodiment, the intrinsic property comprises a compression force related to an electrical transduction in the material, wherein the electrical transduction converts electrical signals into the compression force generating a distributed pressure within a pressure range.

In one embodiment, the predefined pressure range is between 1 mmHg and 150 mmHg.

In another embodiment, the predefined pressure range is between 3 mmHg and 130 mmHg.

In an embodiment, the device further comprises means for attaching the material in order to be attached to the skin over the user body part. In an example, the user body part is an upper or lower extremity of a human and therefore such extremity is covered by the skin. Thus, the device is attached to the skin, in order to transfer (partially or totally) the pressure to the user body part that is covered by the skin. The device is effective even if the user is moving the body part, therefore the device being effective on rest or in movement.

In an embodiment, the means for attaching the material comprises a holder for holding the elastic material to be attached to the skin over the user body part, wherein the holder presses the material against the skin. In this embodiment, the holders is provided with a material comprising a high coefficient of friction, so that the material, after attached to the user body part, is able to transfer the pressure to the user body part. In an embodiment, the holders, provided with the high coefficient of friction, attaches an elastic material to the user body part, wherein after the elastic material is stretched and attached to the user body part, the elastic material is not able to return to its natural condition and is able to transfer the pressure to the user body part.

In an embodiment, the means for attaching the material comprises an adhesive connected to a surface of the material, where in the adhesive is located on at least one portion of the surface. In this embodiment, an adhesive material is connected in one of the surfaces of the material, and therefore this surface is attached to the user body part. This adhesive material covers partially or totally the surface of the material.

In an embodiment, the means for attaching the material comprises an adhesive property comprised in the material, wherein the adhesive property is located on at least one portion of the surface of the material. In this embodiment, the material is any kind of material that comprises the intrinsic property and an adhesive property, and by means of the adhesive property, the device is attached to the user body part.

In an embodiment, the compression force rebalances and realigns the muscle tissues in order to reduce and eventually eliminate myofascial misalignments.

In a further embodiment, the pressure can be adjusted after the device is already in contact with the user body part, wherein the device allows for a better comfort to the user body part. Therefore, the compression force applied by the device is titrated to comfort the user and improves blood circulation.

In an embodiment, the material is suitable to the user body part, wherein the material comprises many degrees of freedom in order to be adapted according to user body part. For example, the device is able to be applied in any of the upper or lower extremities of the human body.

The device applies in the user body part a controlled pressure to have an effective benefit to the acceleration and amplification of strength and functional restoration of at least a user body part. In one embodiment, the pressure is within a predefined pressure range.

In one embodiment, for further control of pressure, the device additionally comprises a component for indication of pressure to be applied by the material to the user body part. In further embodiment, the component for indication of pressure comprises a visual indicator related to the pressure to be applied.

In an embodiment, the visual indicator is positioned in the material, is variable and it deforms itself according to the applied force in at least one extremity of the elastic material, in such a way that it shows different values of pressure for different forces applied. In another embodiment, the visual indicator is positioned in the material, is variable and it deforms itself according to the compression force related to the reaction of chemical reagents, in such a way that it shows different values of pressure for different compression forces. In another embodiment, the visual indicator is positioned in the material, is variable and it deforms itself according to the compression force converted from electrical signals, in such a way that it shows different values of pressure for different compression forces.

In another embodiment, the component comprises an electronic sensor 2 connected to an electronic circuit 3 and positioned in the material. In a further embodiment, the electronic sensor 2 is arranged to detect applied force in at least one extremity of the material. In another embodiment, the electronic sensor 2 is arranged to detect the compression force related to the reaction of chemical reagents. In another embodiment, the electronic sensor 2 is arranged to detect the compression force converted from electrical signals. The electronic circuit 3 is responsible to process the force detected and to display the pressure to be applied by the elastic material. An embodiment of said electronic circuit 3 is shown in FIG. 3.

In an embodiment, the visual indicator is markings on the surface of the material 1 that show different values according to the stretching of elastic material. In another embodiment, the visual indicator is markings on the surface of the material 1 that show different values according to the compression force related to the reaction of chemical reagents. In another embodiment, the visual indicator is markings on the surface of the material 1 that show different values according to the compression force converted from electrical signals.

### EXAMPLE

The example has the only objective of showing one of the innumerous ways to perform the present invention, without limiting the scope of the invention.

The device, in this example, is a soft, conformable, expandable, stretchable wearable, breathable, water-resistant, portable textile as the elastic material. In one embodiments the materials used to fabricate the textile comprises one of knit, crochet, quilt, or cross cross-stitch sleeves gloves or socks, leg-warmers, support hosiery, compressible garments, stretchable, elastics, corset, cotton, polyesters, nylons, neoprene, comprehensible plastics, sarans, and other shearable washable textiles or a combination of these materials.

For the application of the device, the elastic material is stretched resulting in a pressure to contract itself to its original shape. In a portion of the surface of the elastic material an adhesive is connected to attach the device to the user body part.

The device attached to the user body part distributes this pressure to the region where it is attached, resulting in a pressure against the load located in this region, therefore unloading the region and, in this example, reducing the pain.

In this embodiment, the stiffness coefficient of the elastic material provides a compression force related to an applied force in at least one extremity of the elastic material, wherein the compression force generates a distributed pressure within a pressure range. For example, any person is able to stretch the elastic material applying a force in one or in both extremities of the device, and after releasing, the stiffness coefficient generates said compression force. The compression force is directed to the neutral axis of the elastic material. In this sense, the extremities of the device tend to concentrate the strain of the user body part.

The elastic material is disposed in a concentric design to encircle the user body part and include the optional finger or toe holes for added dexterity, safety, and comfort within the targeted restorative pressure range being 3 mmHg to 130 mmHg. After the attaching the device in the user body part, if required the device can be adjusted after the device is already in contact with the user body part to allow a better comfort to the user.

The compression force also allows the distribution of the pressure in a concentric design to encircle the user body part. In this embodiment, this arrangement attenuates loads by concentric nulling of strain-counter strain and limb positioning. In a further embodiment of the example, a plurality of this device is harmonically arranged around the user body part. This force applied by the at least one device induces a gateway rebalancing of myofascial misalignments.

The design-in capacity of the textile material permits the user to then invert the wearable to enshroud grasped, bundled, or intertwined, or twisted “snakes” of cables, cord-like, and other compressible and scatter-prone items, to flexibly bind, secure, or otherwise constrain producing a flexible, compact, malleable, portable pouch to generate a pliable cable management system for packing, storing, and/or transporting of a single and nests of cables, cords and other compressible and scatter-prone items.

The flexible weave is secured and stabilized by the custom manufactured “Tri-glide” and “Bi-glide” scaffolds to dampen vibration and allow for smooth integration of dynamic loading profiles to obliterate latency.

The component for indication of a pressure is a biosensor platform for sensoring at least one of shear forces, electrical, mechanical, optical, audio, tactile, thermal, fluid, magnetic, vibration, conductive, compass-based, and pressure sensors in order to sensor at least one of galvanic skin resistance, heart rate variability, heart rate, blood pressure, temperature, accelerometers, pedometers, sleep, glucose, impedance, pulse oximetry, imaging, ultrasonic sensing, carbon dioxide, carbon monoxide, environmental and chemical exposure, mood, contact or touch, and motion sensors. Furthermore it is designed to accommodate re-expandable or springy, compressible or deformable interfaces capable of adapting to fixed and/or conformable integrated platforms.

The device in this example also comprises integration of iterative components or coding to generate, activate, or actuate gradients, polarizing, fields, or layers to include but not limited to: pneumatic, liquid, gel, electrical, magnetic, chemical, radiant, sonic, coded or software materials or systems.

What is claimed is:

1. A method for promoting functional restoration of a user body part comprising the step of generating a distributed pressure by means of a device to the user body part, wherein said distributed pressure is provided by at least a force applied by the device.

2. The method according to claim 1, wherein the step of generating a distributed pressure comprises:
applying a force in at least one extremity of said device, in order to reach a predefined pressure range; and
attaching the device submitted to the force in the user body part.

3. The method according to claim 2, wherein the step of generating a distributed pressure comprises:
applying the force in at least one extremity of said device, in order to reach the predefined pressure range, and to stretch said device; and
attaching the stretched device submitted to the force in the user body part.

4. The method according to claim 2, wherein the predefined pressure comprises a pressure range of 1 mmHg to 150 mmHg.

5. The method according to claim 2, wherein the predefined pressure comprises a pressure range of 3 mmHg to 130 mmHg.

6. The method according to claim 2, wherein the applying a force in at least one extremity of said device generates a measured pressure, which is identified by a component for indication of pressure.

7. The method according to claim 6, wherein the component for indication is defined by at least one of:
a visual indicator, wherein the visual indicator is positioned in the device and indicates different pressures according to the force applied in at least one extremity of the device; or
an electronic circuit, wherein the electronic circuit is connected to an electronic sensor arranged to detect the applied force in at least one extremity of the device, the electronic circuit process the detected force and displays the pressure in the device.

8. The method according to claim 2, wherein the attaching of the device in the user body part is arranged in a concentric design.

9. The method according to claim 2, wherein a plurality of devices is harmonically arranged to concentric nulling of loads on the user body part.

10. The method according to claim 1, wherein the body part comprises at least one of upper and lower extremities.

11. A device for promoting functional restoration of a user body part, wherein the device comprises a material comprising an intrinsic property for generation of a distributed pressure to the user body part, wherein said distributed pressure is provided by at least a force applied by the material in at least one direction.

12. The device according to claim 11, wherein the material comprises at least one of the following:
a structure comprising at least one of the following: elastic material; knitted material; crocheted material; quilted material; cross cross-stitched sleeve glove or sock; flexible weave; leg-warmer; support hosiery; compressible garment; stretchable material; corset; cotton material; polyester material; nylon material; neoprene material; comprehensible plastic; saran material; and shearable washable textile; and
a configuration of property comprising at least one of the following: wearable, stretchable; water-resistant; breathable; soft; portable; and moldable.

13. The device according to claim 11, wherein the intrinsic property comprises a compression force related to an applied force in at least one extremity of the material, wherein the compression force generates a distributed pressure within a pressure range.

14. The device according to claim 13, wherein the pressure range comprises pressure from 1 mmHg to 150 mmHg.

15. The device according to claim 13, wherein the pressure range comprises pressure from 3 mmHg to 130 mmHg.

16. The device according to claim 11, wherein the device further comprises means for attaching the material to the skin over the user body part.

17. The device according to claim 11, wherein the means for attaching the material to the skin over the user body part comprises at least one of the following:
a holder for holding the material to be attached to the skin over the user body part, wherein the holder presses the material against the skin;
an adhesive connected to a surface of the material, wherein the adhesive is located on at least one portion of the surface; and
an adhesive property comprised in the material, wherein the adhesive property is located on at least one portion of the surface of the material.

18. The device according to claim 11, wherein the device further comprises a component for indication of pressure comprising at least one of:
a visual indicator related to the distributed pressure generated, wherein the visual indicator is positioned in the elastic material and variable according to a applied force in at least one extremity of the elastic material; and
an electronic sensor connected to an electronic circuit and positioned in the elastic material, wherein the electronic sensor is arranged to detect the applied force in at least one extremity of the elastic material.

19. The device according to claim 11, wherein the device is configured to perform a method for promoting functional restoration of a user body part comprising the step of generating a distributed pressure by means of a device to the user body part, wherein said distributed pressure is provided by at least a force applied by the device.
